(19) United States
(12) Patent Application Publication
(10) Pub. No.: US 2012/0220520 A1
(43) Pub. Date: Aug. 30, 2012

(54) BIOAVAILABLE COMBINATIONS FOR HCV TREATMENT

(76) Inventors: Gerben Albert Eleutherius Van't Klooster, Breda (NL); Herman Augustinus De Kock, Arendonk (BE); Pierre Jean-Marie Bernard Raboisson, Rosieres (BE); Christel Florentina E. Van Den Eynde, Begijnendijk (BE)

(21) Appl. No.: 12/445,039

(22) PCT Filed: Jan. 26, 2007
(86) PCT No.: PCT/EP2007/061092

§ 371 Date: Apr. 9, 2009

(30) Foreign Application Priority Data
Oct. 17, 2006 (EP) 06122446.5

(51) Int. Cl. A61K 38/06; A61P 31/14; A61K 31/427; C07D 417/12; A61K 31/4965; A61K 31/4709

(52) U.S. Cl. 514/4.3; 514/255.05; 514/312; 514/365; 548/204

(57) ABSTRACT

The present invention relates to the combination comprising an HCV NS3/4a protease inhibitor and a compound of formula (II). The combination is useful to improve the bioavailability of the HCV NS3/4a protease inhibitor. As such, the combination is useful for treating conditions associated with the Hepatitis C virus in patients. Pharmaceutical compositions and kits comprising this combination, and processes for preparing the combination and the pharmaceutical formulations are also provided.

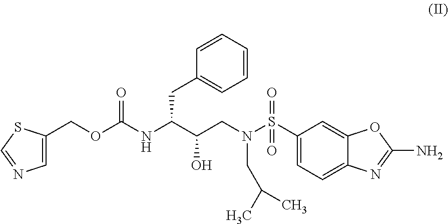

The present invention relates to the combination comprising an HCV NS3/4a protease inhibitor and a compound of formula (II). The combination is useful to improve the bioavailability of the HCV NS3/4a protease inhibitor. As such, the combination is useful for treating conditions associated with the Hepatitis C virus in patients. Pharmaceutical compositions and kits comprising this combination, and processes for preparing the combination and the pharmaceutical formulations are also provided.

Hepatitis C virus is a leading cause of chronic liver disease worldwide and has become a focus of considerable medical research. HCV is a member of the Flaviviridae family of viruses in the hepacivirus genus, and is closely related to the flavivirus genus, which includes a number of viruses implicated in human disease, such as dengue virus and yellow fever virus, and to the animal pestivirus family, which includes bovine viral diarrhea virus (BVDV).

Following the initial acute infection, a majority of infected individuals develop chronic hepatitis because HCV replicates preferentially in hepatocytes but is not directly cytopathic. In particular, the lack of a vigorous T-lymphocyte response and the high propensity of the virus to mutate appear to promote a high rate of chronic infection. Chronic hepatitis can progress to liver fibrosis leading to cirrhosis, end-stage liver disease, and HCC (hepatocellular carcinoma), making it the leading cause of liver transplantations.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

There are 6 major HCV genotypes and more than 50 subtypes, which are differently distributed geographically. HCV type 1 is the predominant genotype in Europe and the US. The extensive genetic heterogeneity of HCV has important diagnostic and clinical implications, perhaps explaining difficulties in vaccine development and the lack of response to therapy.

Current HCV therapies are based on (pegylated) interferon-alpha (IFN-α) in combination with ribavirin. This combination therapy yields a sustained virologic response in more than 40% of patients infected by genotype 1 viruses and about 80% of those infected by genotypes 2 and 3. Beside the limited efficacy on HCV type 1, this combination therapy has significant side effects and is poorly tolerated in many patients. Major side effects include influenza-like symptoms, hematologic abnormalities, and neuropsychiatric symptoms. Hence there is a need for more effective, convenient and better tolerated treatments.

The HCV NS3 serine protease and its associated cofactor, NS4A, mediate a number of proteolytic cleavages of the HCV polyprotein which results in the generation of the HCV replication enzymes. It is thus considered essential for viral replication. As such, interrupting this stage of the viral cycle results in therapeutically active agents. Consequently it is an attractive target for drug discovery.

WO00/059929 discloses macrocyclic compounds active in in-vitro and in cellular assays against the NS3 protease of the hepatitis C virus.

WO02/018369 relates to peptidomimetic compounds useful as protease inhibitors, particularly as serine protease inhibitors and more particularly as hepatitis C NS3 protease inhibitors; intermediates thereto; their preparation including novel steroselective processes to intermediates. The invention is also directed to pharmaceutical compositions and to methods for using the compounds for inhibiting HCV protease or treating a patient suffering from an HCV infection or physiological condition related to the infection. Also provided are pharmaceutical combinations comprising, in addition to one or more HCV serine protease inhibitors, one or more interferons exhibiting anti-HCV activity and/or one or more compounds having anti HCV activity and a pharmaceutically acceptable carrier, and methods for treating or preventing a HCV infection in a patient using the compositions.

WO02/008244 discloses compounds which have HCV protease inhibitory activity as well as methods for preparing such compounds. In another embodiment, the invention discloses pharmaceutical compositions comprising such compounds as well as methods of using them to treat disorders associated with the HCV protease.

WO05/037214 provides compounds as well as compositions, including pharmaceutical compositions, comprising a subject compound. The invention further provides treatment methods, including methods of treating a hepatitis C virus infection and methods of treating liver fibrosis, the methods generally involving administering to an individual in need thereof an effective amount of a subject compound or composition. WO05/095403 provides macrocylic compounds, as well as compositions, including pharmaceutical compositions, comprising a subject compound. The embodiments further provide treatment methods, including methods of treating flaviviral infection, including hepatitis C virus infection and methods of treating liver fibrosis, the methods generally involving administering to an individual in need thereof an effective amount of a subject compound or composition.

A particular group of HCV NS3/4a protease inhibitors is the one comprised by the compounds of formula (I), and the pharmaceutically acceptable salts thereof. These HCV NS3/4a protease inhibitors have been described in WO2005073216 and WO2005073195. Patent applications WO07/014,918, WO07/014,919, WO07/014,926, which are prior art under Art. 54(3) EPC, disclose further examples of compounds falling under formula (I). Certain candidate drugs forming part of this group of HCV inhibitors have been designated to enter into clinical development. These inhibitors have the formula shown hereunder:

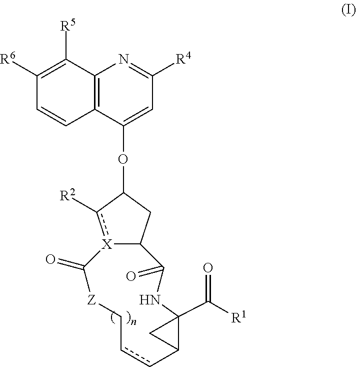

It is known that some drugs are extensively metabolized by the cytochrome P450 system. The cytochrome P450 system is a group of enzymes found in the liver and the gut, which have a number of functions in the human body. One function is the breakdown and clearance of medications and other chemicals.

Metabolization of certain drugs by the cytochrome P450 system frequently results in a drug having unfavourable pharmacokinetics and the need for more frequent and higher doses than are most desirable. Administration of such drug with an agent that inhibits metabolism by the cytochrome P450 system may improve the pharmacokinetics of the drug. This type of applied drug-drug interaction is referred to as “boosting”, i.e. the phenomenon by which at least one of the pharmacokinetic variables of a certain drug is increased. Boosting also supports simplified treatment regimens by the reduction of pill burden and frequency of daily intakes. In this respect, methods for improving the pharmacokinetics of certain drugs have been published, see, e.g., U.S. Pat. No. 6,037,157; D. E. Kempf et al. Antimicrob. Agents Chemother., 41, pp. 654-660 (1997). Further, in US2002/0039998 there is disclosed a method for improving the pharmacokinetics of a drug which is metabolized by cytochrome P450 monooxygenase.

An enantiomer of the compound of formula (II) below and the pharmaceutically acceptable salts thereof are described in WO02/092595, which patent application further discloses the use of the compounds comprised therein as HIV protease inhibitors.

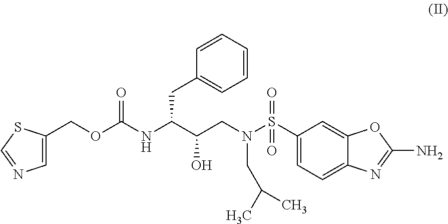

Further, WO05/030194 discloses sulfonamide derivatives, including the racemic of the compound of formula (II), as inhibitors of HCV in mammals.

Patent application WO06/108879, which is prior art under Art. 54(3) EPC, discloses the use of compound of formula (II) and the pharmaceutically acceptable salts thereof as an improver of the pharmacokinetics of a drug, particularly an HIV protease inhibitor, wherein said drug is metabolized by cytochrome P450.

It has been surprisingly found that the bioavailability of certain HCV NS3/4a protease inhibitors is improved when these compounds are combined with the compound of formula (II) or the pharmaceutically acceptable salts thereof.

The combination of one HCV NS3/4a protease inhibitor and a compound of formula (II) is beneficial in that it permits the provision of a therapy to HCV infected patients which is safer, is more effective, and allows a lower therapeutically effective dose of the HCV inhibitor, compared to when such would be administered alone. A lower dose is always desirable in terms of toxicity and pill burden, thereby diminishing the incidence of adverse effects and increasing treatment compliance, respectively. The combination of a HCV NS3/4a protease inhibitor and a compound of formula (II) provides a synergistic effect on the HCV inhibitor upon administration of said combination to a patient in need thereof.

Thus, in one embodiment of the present invention there is provided a combination comprising
(a) an HCV NS3/4a protease inhibitor or a pharmaceutically acceptable salt thereof; wherein the HCV NS3/4a protease inhibitor is metabolized by cytochrome P450; and
(b) a compound of the formula (II),

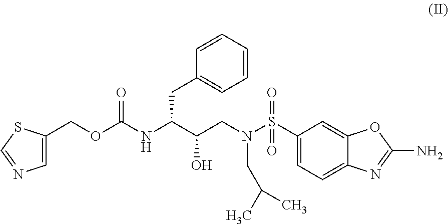

or a pharmaceutically acceptable salt thereof.

The HCV NS3/4a protease inhibitor or a pharmaceutically acceptable salt thereof, party to the combination of the present invention, may be selected from the compounds of WO02/18369 (see, e.g., page 273, lines 9-22 and page 274, line 4 to page 276, line 11), BILN-2061, VX-950, SCH 503034, ITMN-191, or the compound of formula (I)

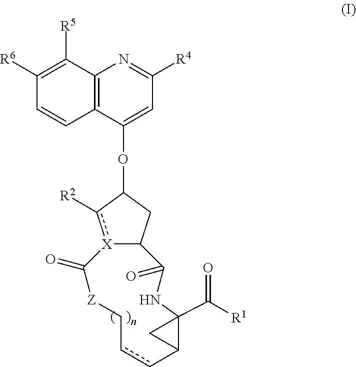

the salts and stereoisomeric forms thereof, wherein
each dashed line (represented by - - - - -) represents an optional double bond;
X is N, CH and where X bears a double bond it is C;
Z is —NR³—, —CR^{3a}R^{3b}—;
R¹ is —OR⁷, —NH—SO₂R⁸;
R² is hydrogen, and where X is C or CH, R² may also be C₁₋₆alkyl;
R³ is hydrogen, C₁₋₆alkyl, C₁₋₆alkoxyC₁₋₆alkyl, C₃₋₇cycloalkyl;
R^{3a} and R^{3b} are hydrogen or C₁₋₆alkyl; or R^{3a} and R^{3b} taken together may form a C₃₋₇cycloalkyl ring;
R⁴ is aryl or Het;
n is 3, 4, 5, or 6;
R⁵ represents hydrogen, halo, C₁₋₆alkyl, hydroxy, C₁₋₆alkoxy, polyhaloC₁₋₆alkyl, phenyl, or Het;
R⁶ represents C₁₋₆alkoxy, mono- or diC₁₋₆alkylamino;
R⁷ is hydrogen; aryl; Het; C₃₋₇cycloalkyl optionally substituted with C₁₋₆alkyl; or C₁₋₆alkyl optionally substituted with C₃₋₇cycloalkyl, aryl or with Het;
R⁸ is aryl; Het; C₃₋₇cycloalkyl optionally substituted with C₁₋₆alkyl; or C₁₋₆alkyl optionally substituted with C₃₋₇cycloalkyl, aryl or with Het;
aryl as a group or part of a group is phenyl optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkyl-amino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkyl-piperazinyl, 4-C₁₋₆alkylcarbonyl-piperazinyl, and morpholinyl; wherein the morpholinyl and piperidinyl groups may be optionally substituted with one or with two C₁₋₆alkyl radicals;
Het as a group or part of a group is a 5 or 6 membered saturated, partially unsaturated or completely unsaturated heterocyclic ring containing 1 to 4 heteroatoms each independently selected from nitrogen, oxygen and sulfur, said heterocyclic ring being optionally condended with a benzene ring; and Het as a whole being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylpiperazinyl, 4-C₁₋₆alkylcarbonylpiperazinyl, and morpholinyl; wherein the morpholinyl and piperidinyl groups may be optionally substituted with one or with two C₁₋₆alkyl radicals.

BILN-2061 has the following structure:

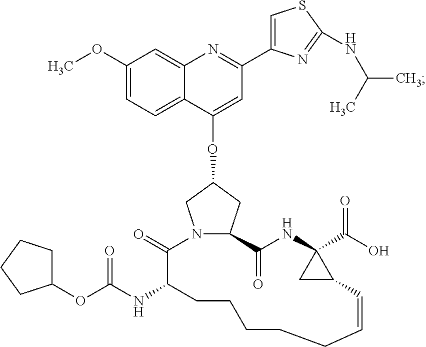

VX-950 has the following structure:

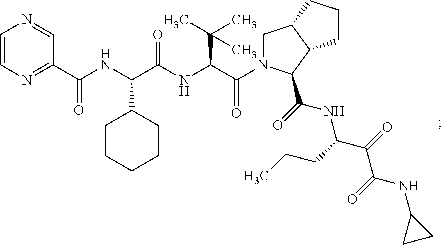

SCH 503034 has the following structure:

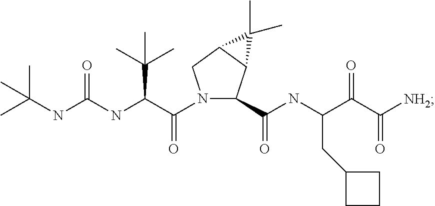

ITMN-191 has the following structure:

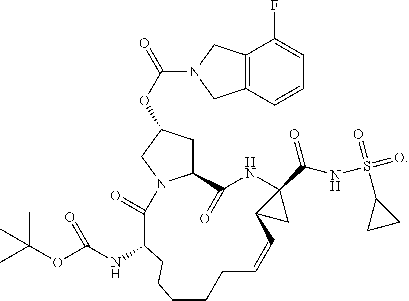

As used in the foregoing and hereinafter, the following definitions apply unless otherwise noted.

Whenever the term “substituted” is used in defining the HCV protease inhibitors of the invention, it is meant to indicate that one or more hydrogens on the atoms mentioned or comprised in the expression using “substituted” is replaced with a selection from the indicated group, provided that the said atoms' normal valency is not exceeded, and that the substitution results in a chemically stable compound, i.e. a compound that maintains its structural and molecular identity in a useful degree of purity through a convenient amount of time. The convenient amount of time will depend on the field of application.

The term halo is generic to fluoro, chloro, bromo and iodo.

The term “polyhaloC₁₋₆alkyl” as a group or part of a group, e.g. in polyhaloC₁₋₆alkoxy, is defined as mono- or polyhalo substituted C₁₋₆alkyl, in particular C₁₋₆alkyl substituted with up to one, two, three, four, five, six, or more halo atoms, such as methyl or ethyl with one or more fluoro atoms, for example, difluoromethyl, trifluoromethyl, trifluoro-ethyl. Preferred is trifluoromethyl. Also included are perfluoroC₁₋₆alkyl groups, which are C₁₋₆alkyl groups wherein all hydrogen atoms are replaced by fluoro atoms, e.g. pentafluoroethyl. In case more than one halogen atom is attached to an alkyl group within the definition of polyhaloC₁₋₆alkyl, the halogen atoms may be the same or different.

As used herein “C₁₋₄alkyl” as a group or part of a group defines straight or branched chain saturated hydrocarbon radicals having from 1 to 4 carbon atoms such as for example methyl, ethyl, 1-propyl, 2-propyl, 1-butyl, 2-butyl, 2-methyl-1-propyl; “C₁₋₆alkyl” encompasses C₁₋₄alkyl radicals and the higher homologues thereof having 5 or 6 carbon atoms such as, for example, 1-pentyl, 2-pentyl, 3-pentyl, 1-hexyl, 2-hexyl, 2-methyl-1-butyl, 2-methyl-1-pentyl, 2-ethyl-1-butyl, 3-methyl-2-pentyl, and the like. Of interest amongst C₁₋₆alkyl is C₁₋₄alkyl.

The term “C₂₋₆alkenyl” as a group or part of a group defines straight and branched chained hydrocarbon radicals having saturated carbon-carbon bonds and at least one double bond, and having from 2 to 6 carbon atoms, such as, for example, ethenyl (or vinyl), 1-propenyl, 2-propenyl (or allyl), 1-butenyl, 2-butenyl, 3-butenyl, 2-methyl-2-propenyl, 2-pentenyl, 3-pentenyl, 2-hexenyl, 3-hexenyl, 4-hexenyl, 2-methyl-2-butenyl, 2-methyl-2-pentenyl and the like. Of interest amongst C₂₋₆alkenyl is C₂₋₄alkenyl.

The term “C₂₋₆alkynyl” as a group or part of a group defines straight and branched chained hydrocarbon radicals having saturated carbon-carbon bonds and at least one triple bond, and having from 2 to 6 carbon atoms, such as, for example, ethynyl, 1-propynyl, 2-propynyl, 1-butynyl, 2-butynyl, 3-butynyl, 2-pentynyl, 3-pentynyl, 2-hexynyl, 3-hexynyl and the like. Of interest amongst C₂₋₆alkynyl is C₂₋₄alkynyl.

C₃₋₇cycloalkyl is generic to cyclopropyl, cyclobutyl, cyclopentyl, cyclohexyl and cycloheptyl.

C₁₋₄alkanediyl defines bivalent straight and branched chain saturated hydrocarbon radicals having from 1 to 4 carbon atoms such as, for example, methylene, ethylene, 1,3-propanediyl, 1,4-butanediyl, 1,2-propanediyl, 2,3-butanediyl, and the like.

C₁₋₆alkanediyl defines bivalent straight and branched chain saturated hydrocarbon radicals having from 1 to 6 carbon atoms such as the radicals exemplified for C₁₋₄alkanediyl, 1,5-pentanediyl, 1,6-hexanediyl, and the like. Of interest amongst C₁₋₆alkanediyl is C₁₋₄alkanediyl.

C₁₋₆alkoxy means C₁₋₆alkyloxy wherein C₁₋₆alkyl is as defined above.

As used herein before, the term (═O) or oxo forms a carbonyl moiety when attached to a carbon atom, a sulfoxide moiety when attached to a sulfur atom and a sulfonyl moiety when two of said terms are attached to a sulfur atom. Whenever a ring or ring system is substituted with an oxo group, the carbon atom to which the oxo is linked is a saturated carbon.

The radical Het is a heterocycle as specified in this specification and claims. Preferred amongst the Het radicals are those that are monocyclic.

Examples of Het comprise, for example, pyrrolidinyl, piperidinyl, morpholinyl, thiomorpholinyl, piperazinyl, pyrrolyl, pyrazolyl, imidazolyl, oxazolyl, isoxazolyl, thiazinolyl, isothiazinolyl, thiazolyl, isothiazolyl, oxadiazolyl, thiadiazolyl, triazolyl (including 1,2,3-triazolyl, 1,2,4-triazolyl), tetrazolyl, furanyl, thienyl, pyridyl, pyrimidyl, pyridazinyl, triazinyl, and the like. Of interest amongst the Het radicals are those which are non-saturated, in particular those having an aromatic character. Of further interest are those Het radicals having one or two nitrogens.

Each of the Het radicals mentioned in this and the following paragraph may be optionally substituted with the number and kind of substituents mentioned in the definitions of the compounds of the present invention or any of the subgroups of compounds of formula (II). Some of the Het radicals mentioned in this and the following paragraph may be substituted with one, two or three hydroxy substituents. Such hydroxy substituted rings may occur as their tautomeric forms bearing keto groups. For example a 3-hydroxypyridazine moiety can occur in its tautomeric form 2H-pyridazin-3-one. Where Het is piperazinyl, it preferably is substituted in its 4-position by a substituent linked to the 4-nitrogen with a carbon atom, e.g. 4-C₁₋₆alkyl, 4-polyhaloC₁₋₆alkyl, C₁₋₆alkoxyC₁₋₆alkyl, C₁₋₆alkylcarbonyl, C₃₋₇cycloalkyl.

Interesting Het radicals comprise, for example pyrrolidinyl, piperidinyl, morpholinyl, thiomorpholinyl, piperazinyl, pyrrolyl, pyrazolyl, imidazolyl, oxazolyl, isoxazolyl, thiazolyl, isothiazolyl, oxadiazolyl, thiadiazolyl, triazolyl (including 1,2,3-triazolyl, 1,2,4-triazolyl), tetrazolyl, furanyl, thienyl, pyridyl, pyrimidyl, pyridazinyl, pyrazolyl, triazinyl, or any of such heterocycles condensed with a benzene ring, such as indolyl, indazolyl (in particular 1H-indazolyl), indolinyl, quinolinyl, tetrahydroquinolinyl (in particular 1,2,3,4-tetrahydroquinolinyl), isoquinolinyl, tetrahydroisoquinolinyl (in particular 1,2,3,4-tetrahydroisoquinolinyl), quinazolinyl, phthalazinyl, benzimidazolyl, benzoxazolyl, benzisoxazolyl, benzothiazolyl, benzoxadiazolyl, benzothiadiazolyl, benzofuranyl, benzothienyl.

The Het radicals pyrrolidinyl, piperidinyl, morpholinyl, thiomorpholinyl, piperazinyl, 4-substituted piperazinyl preferably are linked via their nitrogen atom (i.e. 1-pyrrolidinyl, 1-piperidinyl, 4-thiomorpholinyl, 4-morpholinyl, 1-piperazinyl, 4-substituted 1-piperazinyl).

It should be noted that the radical positions on any molecular moiety used in the definitions may be anywhere on such moiety as long as it is chemically stable.

Radicals used in the definitions of the variables include all possible isomers unless otherwise indicated. For instance pyridyl includes 2-pyridyl, 3-pyridyl and 4-pyridyl; pentyl includes 1-pentyl, 2-pentyl and 3-pentyl.

When any variable occurs more than one time in any constituent, each definition is independent.

The terms “radical(s)”, “substituent(s)” and “variable(s)” are to be interpreted as equivalent unless the context prescribes otherwise.

Whenever used hereinafter, the term “compounds of formula (I)”, “compounds of formula (III)”, “compounds of formula (IV)”, “compounds of formula (V)”, “compounds of formula (VI)”, “the present compounds”, “the compounds of the invention” or similar terms, it is meant to include the compounds of formula (I), (III), (IV), (V), (VI) as appropriate, or any subgroup thereof, the compounds as depicted in Tables 1 or 2, and the prodrugs, stereochemically isomeric forms, racemic mixtures, esters, addition salts, quaternary amines, N-oxides, metal complexes, and metabolites thereof. One embodiment comprises the compounds of formula (I), (III), (IV), (V), (VI), or any subgroup thereof specified herein, as well as the N-oxides, salts, as the possible stereoisomeric forms thereof. Another embodiment comprises the compounds of formula (I), (III), (IV), (V), (VI) or any subgroup thereof specified herein, as well as the salts as the possible stereoisomeric forms thereof.

The compounds of formula (I) have several centers of chirality and exist as stereochemically isomeric forms. The term “stereochemically isomeric forms”, “stereoisomeric forms”, and equivament terminology as used herein defines all the possible compounds made up of the same atoms bonded by the same sequence of bonds but having different three-dimensional structures which are not interchangeable, which the compounds of formula (I) may possess.

With reference to the instances where (R) or (S) is used to designate the absolute configuration of a chiral atom within a substituent, the designation is done taking into consideration the whole compound and not the substituent in isolation.

Unless otherwise mentioned or indicated, the chemical designation of a compound encompasses the mixture of all possible stereochemically isomeric forms, which said compound may possess. Said mixture may contain all diastereomers and/or enantiomers of the basic molecular structure of said compound. All stereochemically isomeric forms of the compounds of the present invention both in pure form or mixed with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of the compounds of the invention can be obtained separately by conventional methods. Appropriate physical separation methods that may advantageously be employed are, for example, selective crystallization and chromatography, e.g. column chromatography.

For some of the compounds of the invention, their prodrugs, N-oxides, salts, solvates, quaternary amines, or metal complexes, and the intermediates used in the preparation thereof, the absolute stereochemical configuration was not experimentally determined. A person skilled in the art is able to determine the absolute configuration of such compounds using art-known methods such as, for example, X-ray diffraction.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmacological Basis of Therapeutics, 8^{th} ed, McGraw-Hill, Int. Ed. 1992, “Biotransformation of Drugs”, p 13-15) describing prodrugs generally is hereby incorporated. Prodrugs preferably have excellent aqueous solubility, increased bioavailability and are readily metabolized into the active inhibitors in vivo. Prodrugs of a compound of the present invention may be prepared by modifying functional groups present in the compound in such a way that the modifications are cleaved, either by routine manipulation or in vivo, to the parent compound.

Preferred are pharmaceutically acceptable ester prodrugs that are hydrolysable in vivo and are derived from those compounds of formula (I) having a hydroxy or a carboxyl group. An in vivo hydrolysable ester is an ester, which is hydrolysed in the human or animal body to produce the parent acid or alcohol. Suitable pharmaceutically acceptable esters for carboxy include C₁₋₆alkoxymethyl esters for example methoxy-methyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyl-oxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyl-oxyethyl which may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxy-methoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

For therapeutic use, salts of the compounds of formula (I) are those wherein the counter-ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable acid and base addition salts as mentioned hereinabove are meant to comprise the therapeutically active non-toxic acid and base addition salt forms which the compounds of formula (I) are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-amino salicylic, pamoic and the like acids.

Conversely said salt forms can be converted by treatment with an appropriate base into the free base form.

The compounds of formula (I) containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “solvate” is used herein to describe a molecular complex comprising i) the compounds of the invention as well as the salts thereof, and ii) one or more pharmaceutically acceptable solvent molecules, for example, ethanol, isopropanol, 1-methoxy-2-propanol, methanol, acetone, dichloromethane, ethylacetate, anisol, tetrahydrofurane, or mesylate. The term “hydrate” is employed when said solvent is water.

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

The N-oxide forms of the present compounds are meant to comprise the compounds of formula (I) wherein one or several nitrogen atoms are oxidized to the so-called N-oxide.

It will be appreciated that the compounds of formula (I) may have metal binding, chelating, complex forming properties and therefore may exist as metal complexes or metal chelates. Such metalated derivatives of the compounds of formula (I) are intended to be included within the scope of the present invention.

Some of the compounds of formula (I) may also exist in their tautomeric form. Such forms although not explicitly indicated in the above formula are intended to be included within the scope of the present invention.

As mentioned above, the compounds of formula (I) have several asymmetric centers. In order to more efficiently refer to each of these asymmetric centers, the numbering system as indicated in the following structural formula will be used.

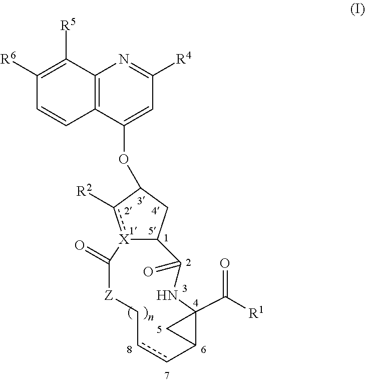

Asymmetric centers are present at positions 1, 4 and 6 of the macrocycle as well as at the carbon atom 3′ in the 5-membered ring, carbon atom 2′ when the R² substituent is C₁₋₆alkyl, and at carbon atom 1′ when X is CH. Z can also encompass an asymmetric center when Z is —CR^{3a}R^{3b}—, and R^{3a} and R^{3b} represent different substituents. Each of these asymmetric centers can occur in their R or S configuration.

The stereochemistry at position 1 preferably corresponds to that of an L-amino acid configuration, i.e. that of L-proline.

When X is CH, the 2 carbonyl groups substituted at positions 1′ and 5′ of the cyclopentane ring preferably are in a trans configuration. The carbonyl substituent at position 5′ preferably is in that configuration that corresponds to an L-proline configuration. The carbonyl groups substituted at positions 1′ and 5′ preferably are as depicted below in the structure of the following formula

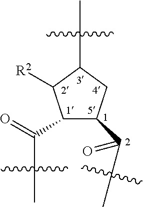

The compounds of formula (I) include a cyclopropyl group as represented in the

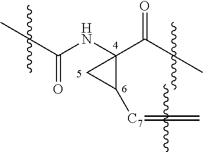

wherein C₇ represents the carbon at position 7 and carbons at position 4 and 6 are asymmetric carbon atoms of the cyclopropane ring. Notwithstanding other possible asymmetric centers at other segments of the compounds of formula (I), the presence of these two asymmetric centers means that the compounds can exist as mixtures of diastereomers, such as the diastereomers of compounds of formula (I) wherein the carbon at position 7 is configured either syn to the carbonyl or syn to the amide as shown below.

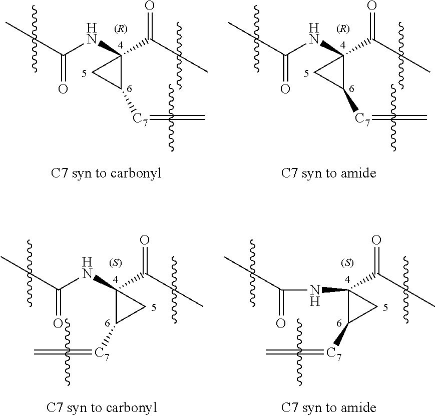

One embodiment concerns compounds of formula (I) wherein the carbon at position 7 is configured syn to the carbonyl. Another embodiment concerns compounds of formula (I) wherein the configuration at the carbon at position 4 is R. A specific subgroup of compounds of formula (I) are those wherein the carbon at position 7 is configured syn to the carbonyl and wherein the configuration at the carbon at position 4 is R.

The compounds of formula (I) may include as well a proline residue (when X is N) or a cyclopentyl or cyclopentenyl residue (when X is CH or C). Preferred are the compounds of formula (I) wherein the substituent at the 1 (or 5′) position and the substituent at position 3′ are in a trans configuration. Of particular interest are the compounds of formula (I) wherein position 1 has the configuration corresponding to L-proline and the substituent at position 3′ is in a trans configuration in respect of position 1. Preferably the compounds of formula (I) have the stereochemistry as indicated in the structures of formulae (I-a) and (I-b) below:

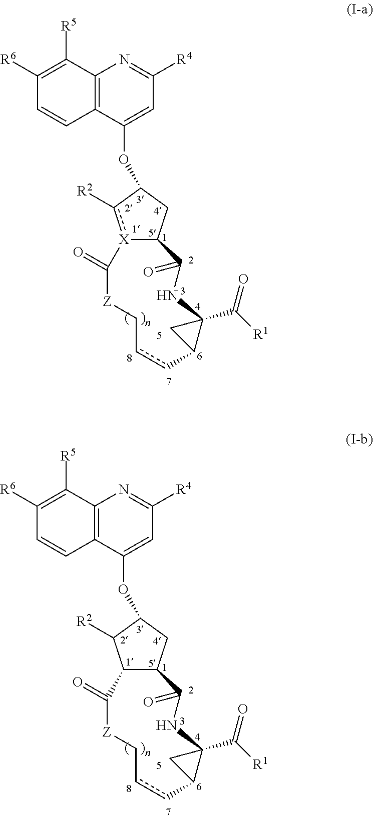

One embodiment of the present invention concerns compounds of formula (I) or of formula (I-a) or of any subgroup of compounds of formula (I), wherein one or more of the following conditions apply:
(a) R² is hydrogen;
(b) X is nitrogen;
(c) a double bond is present between carbon atoms 7 and 8.

One embodiment of the present invention concerns compounds of formula (I) or of formulae (I-a), (I-b), or of any subgroup of compounds of formula (I), wherein one or more of the following conditions apply:
(a) R² is hydrogen;
(b) X is CH;
(c) a double bond is present between carbon atoms 7 and 8.

Particular subgroups of compounds of formula (I) are those represented by the following structural formulae:

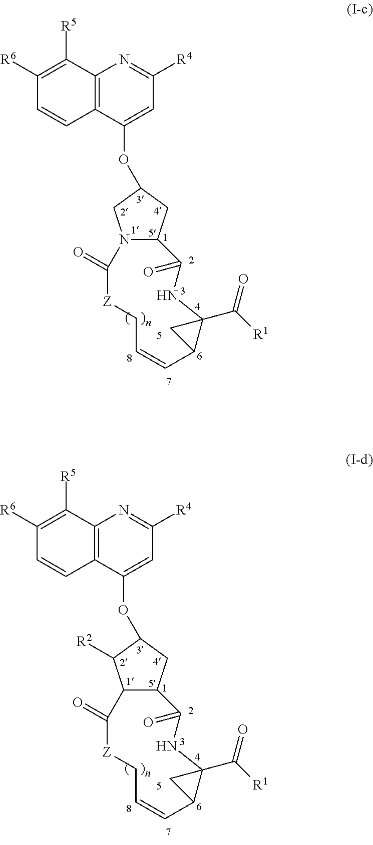

Amongst the compounds of formula (I-c) and (I-d), those having the stereochemical configuration of the compounds of formulae (I-a), and (I-b), respectively, are of particular interest.

The double bond between carbon atoms 7 and 8 in the compounds of formula (I), or in any subgroup of compounds of formula (I), may be in a cis or in a trans configuration. Preferably the double bond between carbon atoms 7 and 8 is in a cis configuration, as depicted in formulae (I-c) and (I-d).

A double bond between carbon atoms 1′ and 2′ may be present in the compounds of formula (I), or in any subgroup of compounds of formula (I), as depicted in formula (I-e) below.

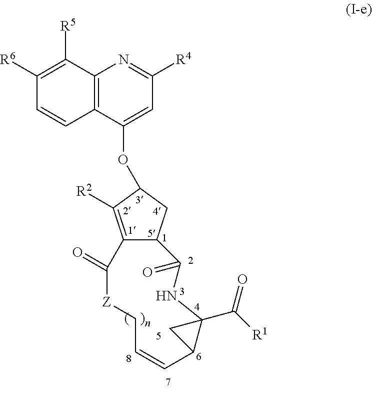

In (I-a), (I-b), (I-c), (I-d), and (I-e), where applicable, X, Z, R¹, R², R³, R⁴, R⁵, and R⁶ are as specified in the definitions of the compounds of formula (I) or in any of the subgroups of compounds of formula (I) specified herein.

It is to be understood that the above defined subgroups of compounds of formulae (I-a), (I-b), (I-c), (I-d), or (I-e), as well as any other subgroup defined herein, are meant to also comprise any prodrugs, N-oxides, addition salts, quaternary amines, metal complexes and stereochemically isomeric forms of such compounds.

When n is 2, the moiety —CH₂— bracketed by “n” corresponds to ethanediyl in the compounds of formula (I) or in any subgroup of compounds of formula (I). When n is 3, the moiety —CH₂— bracketed by “n” corresponds to propanediyl in the compounds of formula (I) or in any subgroup of compounds of formula (I). When n is 4, the moiety —CH₂— bracketed by “n” corresponds to butanediyl in the compounds of formula (I) or in any subgroup of compounds of formula (I). When n is 5, the moiety —CH₂-bracketed by “n” corresponds to pentanediyl in the compounds of formula (I) or in any subgroup of compounds of formula (I). When n is 6, the moiety —CH₂— bracketed by “n” corresponds to hexanediyl in the compounds of formula (I) or in any subgroup of compounds of formula (I). Particular subgroups of the compounds of formula (I) are those compounds wherein n is 4 or 5.

Embodiments of the invention are compounds of formula (I) or any of the subgroups of compounds of formula (I) wherein
(a) R¹ is —OR⁷, in particular wherein R⁷ is C₁₋₆alkyl, such as methyl, ethyl, or tert-butyl and most preferably where R⁷ is hydrogen;
(b) R¹ is —NHS(═O)₂R⁸, in particular wherein R⁸ is C₁₋₆alkyl, C₃-C₇cycloalkyl, or aryl, e.g. wherein R⁸ is methyl, cyclopropyl, or phenyl; or
(c) R¹ is —NHS(═O)₂R⁸, in particular wherein R⁸ is C₃-C₇ cycloalkyl substituted with C₁₋₆alkyl, preferably wherein R⁸ is cyclopropyl, cyclobutyl, cyclopentyl, or cyclohexyl, any of which is substituted with C₁₋₄alkyl, i.e. with methyl, ethyl, propyl, isopropyl, butyl, tert-butyl, or isobutyl.

Further embodiments of the invention are compounds of formula (I) or any of the subgroups of compounds of formula (I) wherein R¹ is —NHS(═O)₂R⁸, in particular wherein R⁸ is cyclopropyl substituted with C₁₋₄alkyl, i.e. with methyl, ethyl, propyl, or isopropyl.

Further embodiments of the invention are compounds of formula (I) or any of the subgroups of compounds of formula (I) wherein R¹ is —NHS(═O)₂R⁸, in particular wherein R⁸ is 1-methylcyclopropyl.

Further embodiments of the invention are compounds of formula (I) or any of the subgroups of compounds of formula (I) wherein
(a) R² is hydrogen;
(b) R² is C₁₋₆alkyl, preferably methyl.

Embodiments of the invention are compounds of formula (I) or any of the subgroups of compounds of formula (I) wherein
(a) X is N, C (X being linked via a double bond) or CH (X being linked via a single bond) and R² is hydrogen;
(b) X is C (X being linked via a double bond) and R² is C₁₋₆alkyl, preferably methyl.

One embodiment of the invention encompasses compounds of formula (I) or any of the subgroups of compounds of formula (I) wherein
(a) Z is —NR³—, and R³ is hydrogen, C₁₋₆alkyl, C₁₋₆alkoxyC₁₋₆alkyl, or C₃₋₇cycloalkyl; or
(b) Z is —CR^{3a}R^{3b}—; and R^{3a} and R^{3b} are, each independently, hydrogen or C₁₋₆alkyl; or R^{3a} and R^{3b} taken together form a C₃₋₇cycloalkyl ring.

Further embodiments of the invention are compounds of formula (I) or any of the subgroups of compounds of formula (I) wherein Z is —NR³—, and
(a) R³ is hydrogen;
(b) R³ is C₁₋₆alkyl; or
(c) R³ is C₁₋₆alkoxyC₁₋₆alkyl or C₃₋₇cycloalkyl.

Further embodiments of the invention are compounds of formula (I) or any of the subgroups of compounds of formula (I) wherein Z is —CR^{3a}R^{3b}—, and
(a) R^{3a} is hydrogen and R^{3b} is C₁₋₆alkyl;
(b) R^{3a} and R^{3b} are both hydrogen;
(c) R^{3a} and R^{3b} are both C₁₋₆alkyl; or
(d) R^{3a} and R^{3b} taken together form a C₃₋₇cycloalkyl ring.

Preferred embodiments of the invention are compounds of formula (I) or any of the subgroups of compounds of formula (I) wherein Z is —NR³—, and R³ is hydrogen, or C₁₋₆alkyl, more preferably hydrogen or methyl.

Further preferred embodiments of the invention are compounds of formula (I) or of any subgroup of compounds of formula (I), wherein Z is —CR^{3a}R^{3b}—, and R^{3a} and R^{3b} are both hydrogen.

Embodiments of the invention are compounds of formula (I) or any of the subgroups of compounds of formula (I) wherein R⁴ is aryl or Het, each independently, optionally substituted with any of the substituents of Het or aryl mentioned in the definitions of the compounds of formula (I) or of any of the subgroups of compounds of formula (I); or specifically said aryl or Het being each, independently, optionally substituted with C₁₋₆alkyl, halo, amino, mono- or diC₁₋₆alkylamino, pyrrolidinyl, piperidinyl, morpholinyl, piperazinyl, 4-C₁₋₆alkylpiperazinyl; and wherein the morpholinyl and piperidinyl groups may optionally substituted with one or two C₁₋₆alkyl radicals;

Embodiments of the invention are compounds of formula (I) or any of the subgroups of compounds of formula (I) wherein R⁴ is a radical

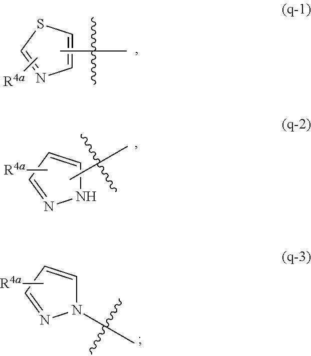

or, in particular, wherein R⁴ is selected from the group consisting of:

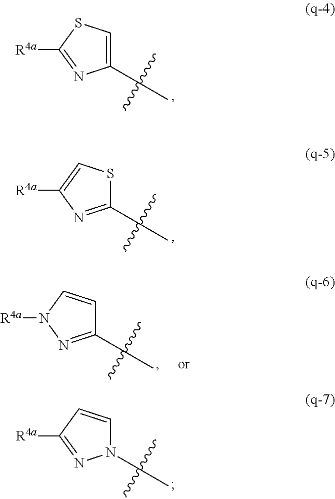

wherein, where possible a nitrogen may bear an R^{4a} substituent or a link to the remainder of the molecule; each R^{4a} in any of the R⁴ substituents may be selected from those mentioned as possible substituents on Het, as specified in the definitions of the compounds of formula (I) or of any of the subgroups of compounds of formula (I);

more specifically each R^{4a} may be hydrogen, halo, C₁₋₆alkyl, amino, or mono- or di-C₁₋₆alkylamino, pyrrolidinyl, piperidinyl, morpholinyl, piperazinyl, 4-C₁₋₆alkyl-piperazinyl; and wherein the morpholinyl and piperidinyl groups may optionally substituted with one or two C₁₋₆alkyl radicals;

more specifically each R^{4a} is, each independently, hydrogen, halo, C₁₋₆alkyl, amino, or mono- or di-C₁₋₆alkylamino;

and where R^{4a} is substituted on a nitrogen atom, it preferably is a carbon containing substituent that is connected to the nitrogen via a carbon atom or one of its carbon atoms; and wherein in that instance R^{4a} preferably is C₁₋₆alkyl.

Embodiments of the invention are compounds of formula (I) or any of the subgroups of compounds of formula (I) wherein R⁴ is phenyl or pyridiyl (in particular 4-pyridyl) which each may be substituted with 1, 2 or 3 substituents selected from those mentioned for aryl in the definitions of the compounds of formula (I) or of any of the subgroups thereof. In particular said phenyl or pyridyl is substituted with 1-3 (or with 1-2, or with one) substituent or substituents selected from halo, C₁₋₆alkyl or C₁₋₆alkoxy.

Embodiments of the invention are compounds of formula (I) or any of the subgroups of compounds of formula (I) wherein R⁵ is halo, or C₁₋₆alkyl, preferably methyl, ethyl, isopropyl, tert-butyl, fluoro, chloro, or bromo. include poluhaloC₁₋₆alkyl

Embodiments of the invention are compounds of formula (I) or any of the subgroups of compounds of formula (I) wherein R⁶ is C₁₋₆alkoxy or diC₁₋₆alkylamino; preferably R⁶ is methoxy or dimethylamino; more preferably R⁶ is methoxy.

The compounds of formula (I) or any of the subgroups of compounds of formula (I) may be prepared according to any one of the methods provided in WO05/073195, WO05/073216.

Compounds of formula (I) may be converted into each other following art-known functional group transformation reactions, comprising those described hereinafter.

A number of the intermediates used to prepare the compounds of formula (I) are known compounds or are analogs of known compounds, which can be prepared following modifications of art-known methodologies readily accessible to the skilled person, including amongst other the methods provided in WO05/073195, WO05/073216.

The compounds of formula (I) may be converted to the corresponding N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert-butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Pure stereochemically isomeric forms of the compounds of formula (I) may be obtained by the application of art-known procedures. Diastereomers may be separated by physical methods such as selective crystallization and chromatographic techniques, e.g., counter-current distribution, liquid chromatography and the like.

The compounds of formula (I) may be obtained as racemic mixtures of enantiomers which can be separated from one another following art-known resolution procedures. The racemic compounds of formula (I), which are sufficiently basic or acidic may be converted into the corresponding diastereomeric salt forms by reaction with a suitable chiral acid, respectively chiral base. Said diastereomeric salt forms are subsequently separated, for example, by selective or fractional crystallization and the enantiomers are liberated therefrom by alkali or acid. An alternative manner of separating the enantiomeric forms of the compounds of formula (I) involves liquid chromatography, in particular liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound may be synthesized by stereospecific methods of preparation. These methods may advantageously employ enantiomerically pure starting materials.

In one embodiment of the present invention there is provided a combination comprising
(a) an HCV NS3/4a protease inhibitor of the formula (III)

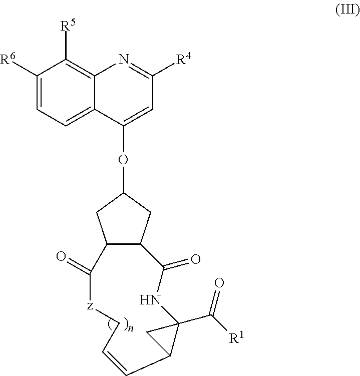

or a pharmaceutically acceptable salt thereof, wherein R¹; Z; R⁴, R⁵, R⁶, n are as recited herein; and
(b) a compound of the formula (II)

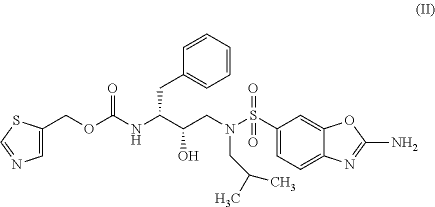

or a pharmaceutically acceptable salt thereof.

The HCV NS3/4a protease inhibitor of the formula (III) can be selected from any one of the following compounds of Table 1.

TABLE 1

| Compound | |
|---|---|
| nr. | Structural formula |
| 1 | 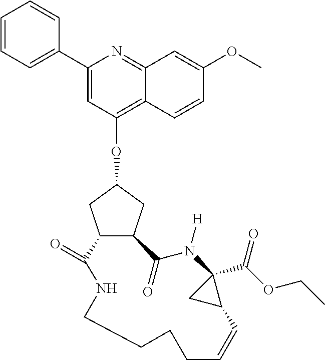 |
| 2 | 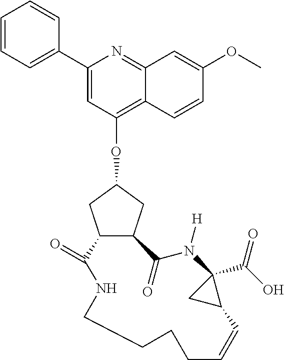 |
| 3 | 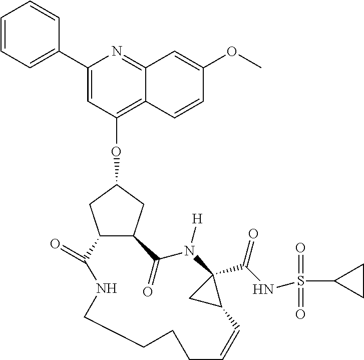 |
| 4 | 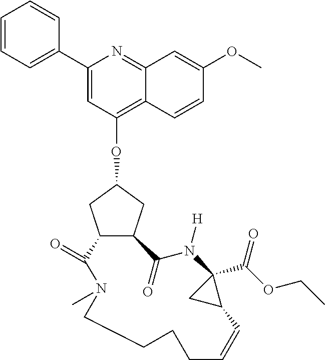 |
| 5 | 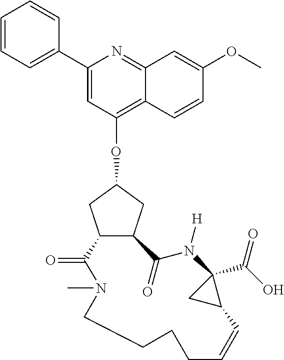 |
| 6 | 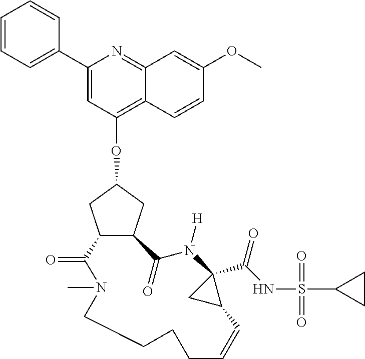 |
| 7 | 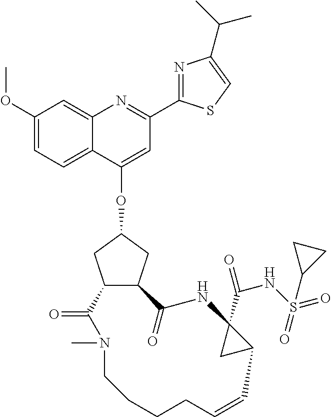 |
| 8 | 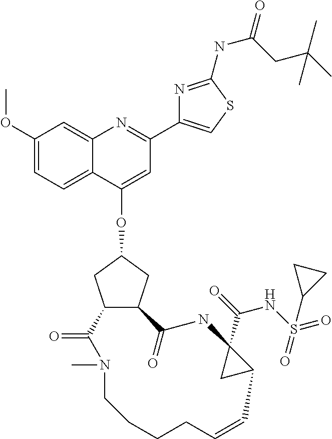 |
| 9 | 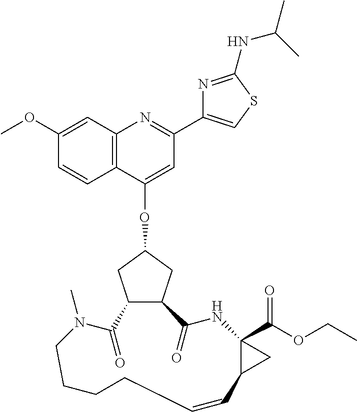 |
| 10 | 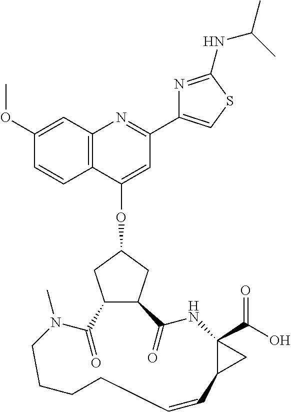 |
| 11 | 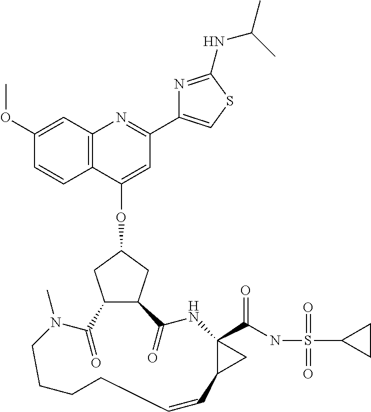 |
| 12 | 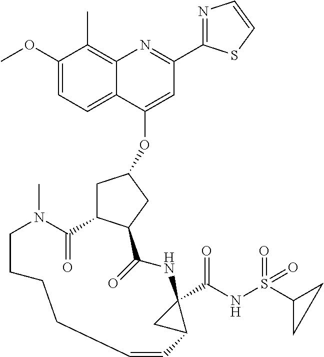 |
| 13 | 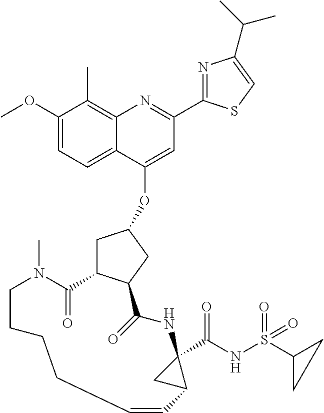 |
| 14 | 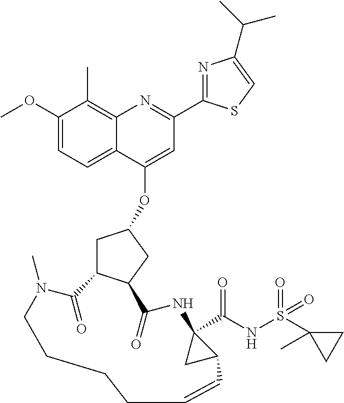 |
| 15 | 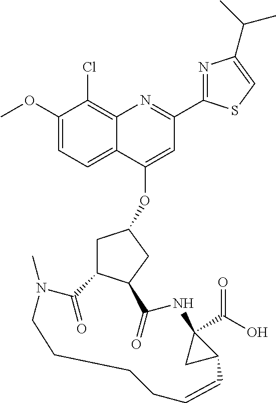 |
| 16 | 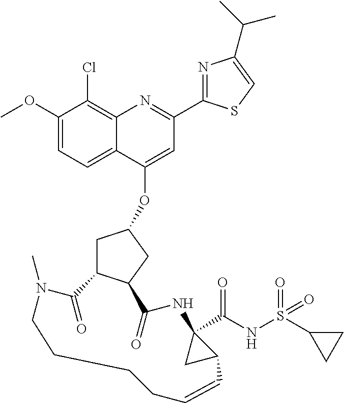 |
| 17 | 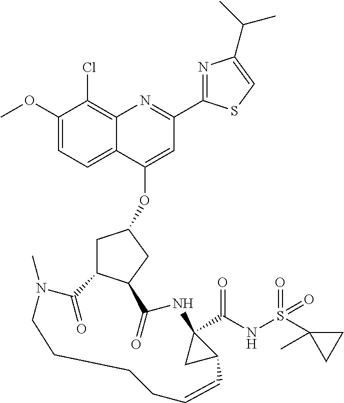 |

In a preferred embodiment of the present invention there is provided a combination comprising (a) an HCV NS3/4a protease inhibitor of the formula (III), or a pharmaceutically acceptable salt thereof; and (b) a compound of the formula (II), or a pharmaceutically acceptable salt thereof; wherein the HCV NS3/4a protease inhibitor of formula (III) is selected from

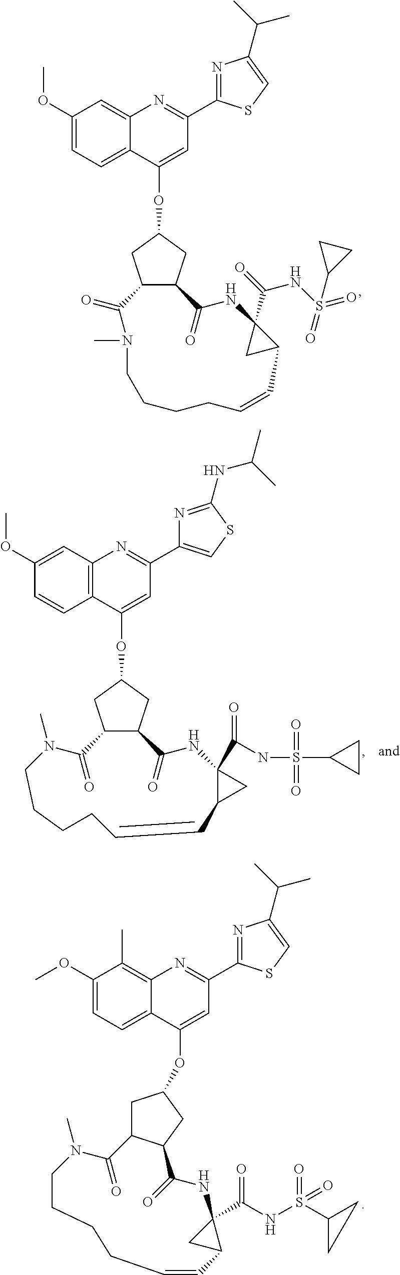

In one embodiment of the present invention there is provided a combination comprising
(c) an HCV NS3/4a protease inhibitor of the formula (IV)

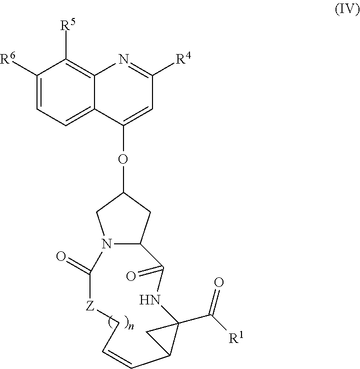

or a pharmaceutically acceptable salt thereof, wherein R¹; Z; R⁴, R⁵, R⁶, n are as recited herein; and
(d) a compound of the formula (II)

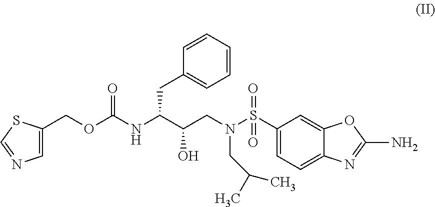

or a pharmaceutically acceptable salt thereof.

The HCV NS3/4a protease inhibitor of the formula (IV) can be selected from any one of the following compounds of Table 2.

TABLE 2

| Com- | |
|---|---|
| pound | |
| nr | |
| 18 | 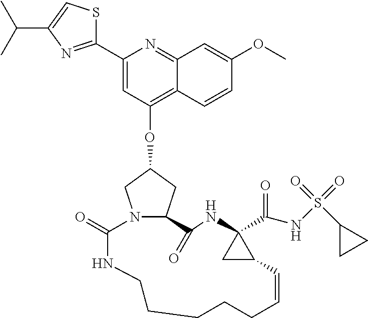 |
| 19 | 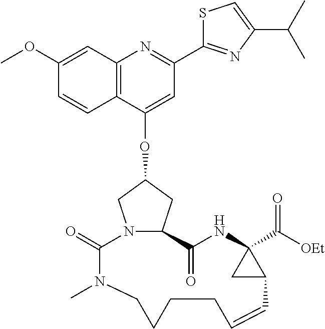 |
| 20 | 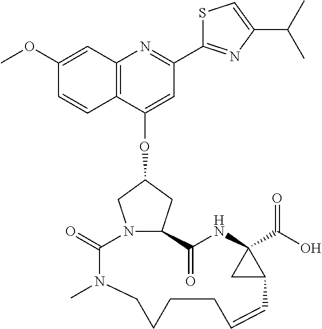 |
| 21 | 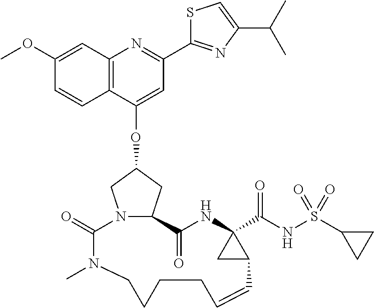 |
| 22 | 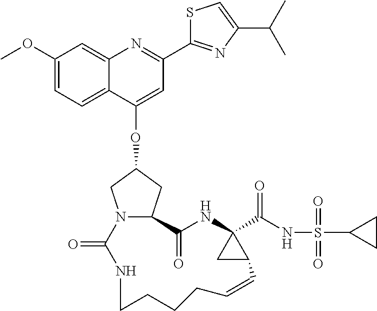 |
| 23 | 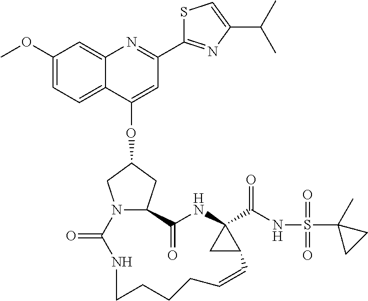 |
| 24 | 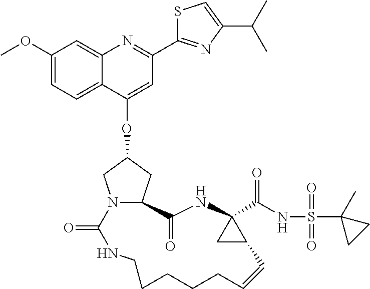 |
| 25 | 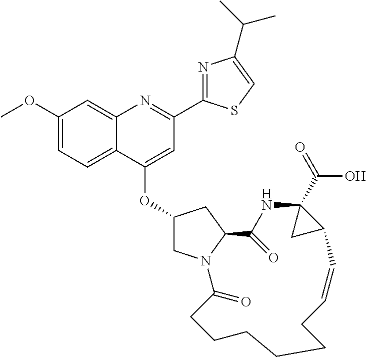 |
| 26 | 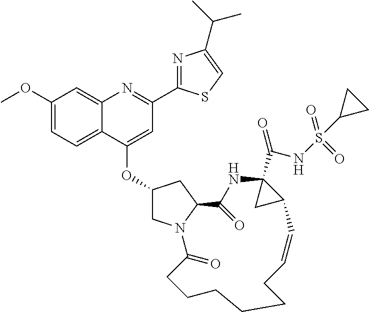 |
| 27 | 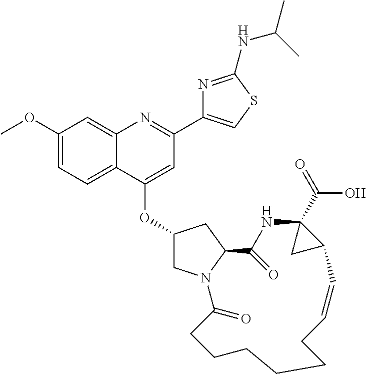 |
| 28 | 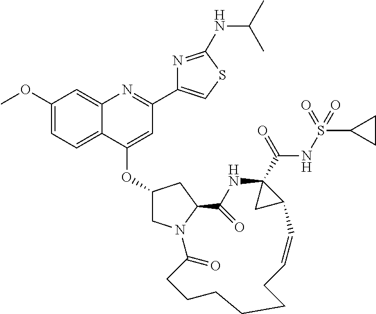 |
| 29 | 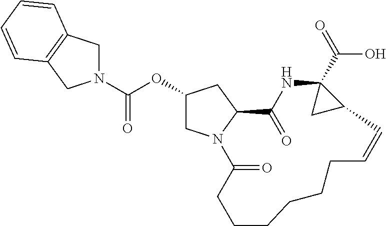 |
| 30 | 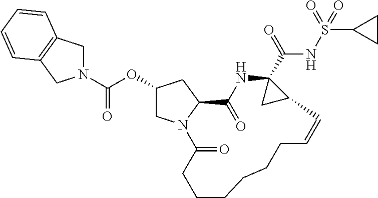 |
| 31 | 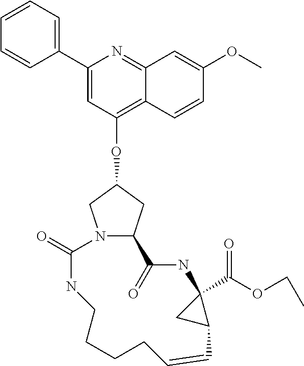 |
| 32 | 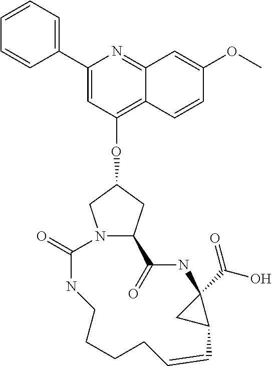 |

In a preferred embodiment of the present invention there is provided a combination comprising (a) an HCV NS3/4a protease inhibitor of the formula (IV), or a pharmaceutically acceptable salt thereof; and (b) a compound of the formula (II), or a pharmaceutically acceptable salt thereof; wherein the HCV NS3/4a protease inhibitor of formula (IV) is selected from

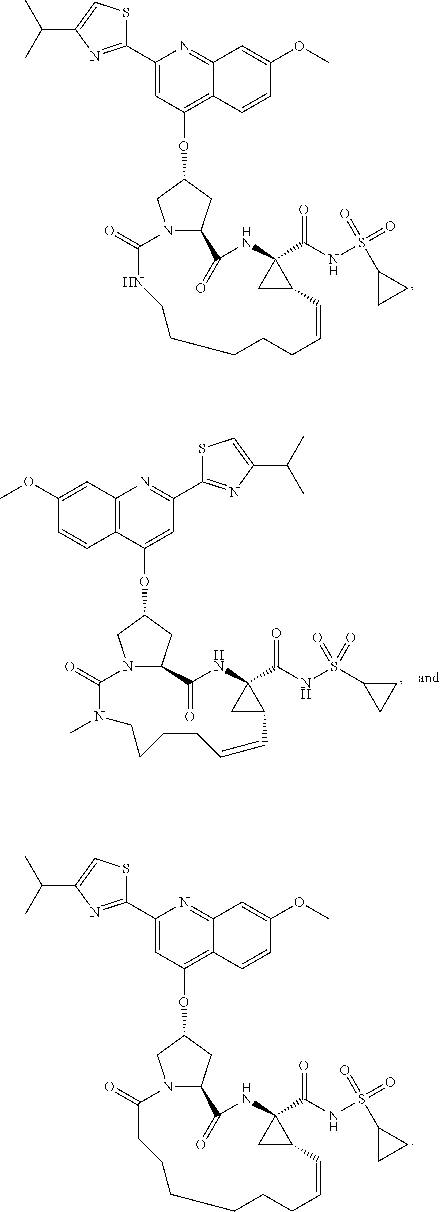

The compounds of formula (I), pharmaceutically acceptable salts thereof, and methods for their preparation are described in WO2005073216, WO2005073195.

Methods for the preparation of the compound of formula (II) and pharmaceutically acceptable salts thereof are described in WO02/092595.

In one embodiment of the invention, the combination as disclosed herein, further comprises an additional HCV antiviral selected from HCV polymerase inhibitors, NM283, R803, JTK-109 and JTK-003; HCV proteases (NS2-NS3 and NS3-NS4A) inhibitors, the compounds of WO02/18369 (see, e.g., page 273, lines 9-22 and page 274, line 4 to page 276, line 11), BILN-2061, VX-950, SCH 503034; inhibitors of other targets in the HCV life cycle, including helicase, and metalloprotease inhibitors, ISIS-14803; immunomodulatory agents such as, α-, β-, and γ-interferons, pegylated derivatized interferon-α compounds, compounds that stimulate the synthesis of interferon in cells, interleukins, compounds that enhance the development of type 1 helper T cell response, and thymosin; other antiviral agents such as ribavirin, amantadine, and telbivudine, inhibitors of internal ribosome entry, broad-spectrum viral inhibitors, such as IMPDH inhibitors (e.g., compounds of U.S. Pat. No. 5,807,876, U.S. Pat. No. 6,498,178, U.S. Pat. No. 6,344,465, U.S. Pat. No. 6,054,472, WO97/40028, WO98/40381, WO00/56331, and mycophenolic acid and derivatives thereof, and including, but not limited to VX-950, VX-497, VX-148, and/or VX-944); or combinations of any of the above.

In one embodiment of the present invention there is provided a process for preparing a combination as described herein, comprising the step of combining an HCV NS3/4a protease inhibitor or a pharmaceutically acceptable salt thereof, and a compound of formula (II) or a pharmaceutically acceptable salt thereof. An alternative embodiment of this invention provides a process wherein the combination comprises one or more additional agent as described herein.

The combinations of the present invention may be used as medicaments. Said use as a medicine or method of treatment comprises the systemic administration to HCV-infected subjects of an amount effective to combat the conditions associated with HCV and other pathogenic flavi- and pestiviruses. Consequently, the combinations of the present invention can be used in the manufacture of a medicament useful for treating, preventing or combating infection or disease associated with HCV infection in a mammal, in particular for treating conditions associated with HCV and other pathogenic flavi- and pestiviruses.

In one embodiment of the present invention there is provided a pharmaceutical composition comprising a combination according to any one of the embodiments described herein and one or more pharmaceutically acceptable excipients. In particular, the present invention provides a pharmaceutical composition comprising (a) a therapeutically effective amount of an HCV NS3/4a protease inhibitor or a pharmaceutically acceptable salt thereof, (b) a therapeutically effective amount of a compound of formula (II) or a pharmaceutically acceptable salt thereof, and (c) a pharmaceutically acceptable excipient. Optionally, the pharmaceutical composition further comprises an additional agent selected from an HCV polymerase inhibitor, an HCV protease inhibitor, an inhibitor of another target in the HCV life cycle, and immunomodulatory agent, an antiviral agent, and combinations thereof.

As used herein, the term “composition” is intended to encompass a product comprising the specified ingredients, as well as any product which results, directly or indirectly, from the combination of the specified ingredients.

The term “therapeutically effective amount” as used herein means that amount of active compound or component or pharmaceutical agent that elicits the biological or medicinal response in a tissue, system, animal or human that is being sought, in the light of the present invention, by a researcher, veterinarian, medical doctor or other clinician, which includes alleviation of the symptoms of the disease being treated. Since the instant invention refers to combinations comprising two or more agents, the “therapeutically effective amount” is that amount of the agents taken together so that the combined effect elicits the desired biological or medicinal response. For example, the therapeutically effective amount of a composition comprising (a) the HCV NS3/4a protease inhibitor and (b) the compound of formula (II), would be the amount of the HCV NS3/4a protease inhibitor and the amount of the compound of formula (II) that when taken together have a combined effect that is therapeutically effective.

The pharmaceutical composition can be prepared in a manner known per se to one of skill in the art. For this purpose, at least one of an HCV NS3/4a protease inhibitor, and a compound of formula (II), together with one or more solid or liquid pharmaceutical excipients and, if desired, in combination with other pharmaceutical active compounds, are brought into a suitable administration form or dosage form which can then be used as a pharmaceutical in human medicine or veterinary medicine.

In one embodiment the combinations of the present invention may also be formulated as a combined preparation for simultaneous, separate or sequential use in HCV therapy. In such a case, the HCV NS3/4a protease inhibitor is formulated in a pharmaceutical composition containing other pharmaceutically acceptable excipients, and the compound of formula (II) is formulated separately in a pharmaceutical composition containing other pharmaceutically acceptable excipients. Conveniently, these two separate pharmaceutical compositions can be part of a kit for simultaneous, separate or sequential use.

Thus, the individual components of the combination of the present invention can be administered separately at different times during the course of therapy or concurrently in divided or single combination forms. The present invention is therefore to be understood as embracing all such regimes of simultaneous or alternating treatment and the term “administering” is to be interpreted accordingly. In a preferred embodiment, the separate dosage forms are administered about simultaneously.

The compositions or products comprising a combination of the present invention, whether co-formulated in a single formulation or formulated for simultaneous, separate or sequential use, may be administered orally (including suspensions, capsules, tablets, sachets, solutions, suspensions, emulsions), sublingually, parenterally (including subcutaneous injections, intravenous, intramuscular, intradermal injection or infusion techniques), by inhalation spray (including nasal sprays), topically, rectally (including suppositories), vaginally, via an implanted reservoir, in dosage unit formulations containing conventional non-toxic pharmaceutically acceptable carriers, adjuvants and vehicles.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

The oral administration of a combination of the present invention is suitably accomplished by uniformly and intimately blending together a suitable amount of each component in the form of a powder, optionally also including a finely divided solid carrier, and encapsulating the blend in, for example, a hard gelatin capsule. The solid carrier can include one or more substances which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Oral administration of a combination of the present invention can also be accomplished by preparing capsules or tablets containing the desired amount of the HCV NS3/4a protease inhibitor only, optionally blended with a solid carrier as described above, and capsules containing the desired amount of the compound of formula (II) only. Compressed tablets containing the HCV NS3/4a protease inhibitor can be prepared by uniformly and intimately mixing the active ingredient with a solid carrier such as described above to provide a mixture having the necessary compression properties, and then compacting the mixture in a suitable machine to the shape and size desired. Molded tablets maybe made by molding in a suitable machine, a mixture of powdered the HCV NS3/4a protease inhibitor of formula (II) moistened with an inert liquid diluent. Oral administration can also be accomplished by preparing compressed or molded tablets containing the HCV NS3/4a protease inhibitor of formula (II) as just described, the tablets of suitable size for insertion into standard capsules (e.g., hard gelatin capsules), and then inserting the tablets into capsules containing a suitable amount of compound of formula (II) powder.

For subcutaneous or intravenous administration, the active components of the compositions, if desired with the substances customary therefore such as solubilizers, emulsifiers or further auxiliaries, are brought into solution, suspension, or emulsion.

The components of the compositions can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the components of the compositions or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compounds in a concentration from approximately 0.1 to 50%, in particular from approximately 0.3 to 3% by weight.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract may be effected in a rectal suppository formulation (see below) or in a suitable enema formulation. Topically-transdermal patches may also be used.

For topical applications, the pharmaceutical compositions may be formulated in a suitable ointment containing the active components suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as 30 benzylalkonium chloride. Alternatively, for ophthalmic uses, the pharmaceutical compositions may be formulated in an ointment such as petrolatum.

When rectally administered in the form of suppositories, these formulations may be prepared by mixing the individual components of a composition according to the invention with a suitable non-irritating excipient, such as cocoa butter, synthetic glyceride esters or polyethylene glycols, which are solid at ordinary temperatures, but liquidify and/or dissolve in the rectal cavity to release the drug.

In another embodiment of the method of the invention, the administration may be performed with food (e.g., a high-fat meal) or without food. The term “with food” means the consumption of a meal either during or no more than about one hour before or after administration of a one or both components of the combination according to the invention.

In one embodiment, the combination of the present invention contains an amount of a compound of formula (II), or a pharmaceutically acceptable salt thereof, which is sufficient to clinically improve the bioavailability of the HCV NS3/4a protease inhibitor relative to the bioavailability when said HCV NS3/4a protease inhibitor is administered alone.

In another embodiment, the combination of the present invention contains an amount of the compound of formula (II), or a pharmaceutically acceptable salt thereof, which is sufficient to increase at least one of the pharmacokinetic variables of the HCV NS3/4a protease inhibitor selected from t_{1/2}, Cₘᵢₙ, Cₘₐₓ, Cₛₛ, AUC at 12 hours, or AUC at 24 hours, relative to said at least one pharmacokinetic variable when the HCV NS3/4a protease inhibitor is administered alone.

A further embodiment relates to a method for improving the bioavailability of a HCV NS3/4a protease inhibitor comprising administering to an individual in need of such improvement a combination as defined herein, comprising a therapeutically effective amount of each component of said combination.

In a further embodiment, the invention relates to the use of the compound of formula (II) or a pharmaceutically acceptable salt thereof, as an improver of at least one of the pharmacokinetic variables of a HCV NS3/4a protease inhibitor selected from t_{1/2}, Cₘᵢₙ, Cₘₐₓ, Cₛₛ, AUC at 12 hours, or AUC at 24 hours; with the proviso that said use is not practised in the human or animal body.

The term “individual” as used herein refers to an animal, preferably a mammal, most preferably a human, who has been the object of treatment, observation or experiment.

Bioavailability is defined as the fraction of administered dose reaching systemic circulation. t_{1/2} represents the half life or time taken for the plasma concentration to fall to half its original value. Cₛₛ is the steady state concentration, i.e. the concentration at which the rate of input of drug equals the rate of elimination. Cₘᵢₙ is defined as the lowest (minimum) concentration measured during the dosing interval. Cₘₐₓ, represents the highest (maximum) concentration measured during the dosing interval. AUC is defined as the area under the plasma concentration-time curve for a defined period of time.

The combinations of this invention can be administered to humans in dosage ranges specific for each component comprised in said combinations. The components comprised in said combinations can be administered together or separately. The NS3/4a protease inhibitors, and the compound of formula (II) or a pharmaceutically acceptable salt or ester thereof, may have dosage levels of the order of 0.02 to 5.0 grams-per-day.

When the HCV NS3/4a protease inhibitor and the compound of formula (II) are administered in combination, the weight ratio of the HCV NS3/4a protease inhibitor to the compound of formula (II) is suitably in the range of from about 40:1 to about 1:15, or from about 30:1 to about 1:15, or from about 15:1 to about 1:15, typically from about 10:1 to about 1:10, and more typically from about 8:1 to about 1:8. Also useful are weight ratios of the HCV NS3/4a protease inhibitors to the compound of formula (II) ranging from about 6:1 to about 1:6, or from about 4:1 to about 1:4, or from about 3:1 to about 1:3, or from about 2:1 to about 1:2, or from about 1.5:1 to about 1:1.5. In one aspect, the amount by weight of the HCV NS3/4a protease inhibitors is equal to or greater than that of the compound of formula (II), wherein the weight ratio of the HCV NS3/4a protease inhibitor to the compound of formula (II) is suitably in the range of from about 1:1 to about 15:1, typically from about 1:1 to about 10:1, and more typically from about 1:1 to about 8:1. Also useful are weight ratios of the HCV NS3/4a protease inhibitor to the compound of formula (II) ranging from about 1:1 to about 6:1, or from about 1:1 to about 5:1, or from about 1:1 to about 4:1, or from about 3:2 to about 3:1, or from about 1:1 to about 2:1 or from about 1:1 to about 1.5:1.

According to one embodiment, the HCV NS3/4a protease inhibitor and the compound of formula (II) may be co-administered once or twice a day, once, twice, three, four, fives or six times a week, preferably orally, wherein the amount of the HCV NS3/4a protease inhibitor per dose is from about 10 to about 2500 mg, and the amount of the compound of formula (II) per dose is from 10 to about 2500 mg. In another embodiment, the amounts per dose for once or twice daily co-administration are from about 50 to about 1500 mg of the HCV NS3/4a protease inhibitor and from about 50 to about 1500 mg of the compound of formula (II). In still another embodiment, the amounts per dose for the daily or weekly co-administration are from about 100 to about 1000 mg of the HCV NS3/4a protease inhibitor and from about 100 to about 800 mg of the compound of formula (II). In yet another embodiment, the amounts per dose for the daily or weekly co-administration are from about 150 to about 800 mg of the HCV NS3/4a protease inhibitor and from about 100 to about 600 mg of the compound of formula (II). In yet another embodiment, the amounts per dose for the daily or weekly co-administration are from about 200 to about 600 mg of the HCV NS3/4a protease inhibitor and from about 100 to about 400 mg of the compound of formula (II). In yet another embodiment, the amounts per dose for the daily or weekly co-administration are from about 200 to about 600 mg of the HCV NS3/4a protease inhibitor and from about 20 to about 300 mg of the compound of formula (II). In yet another embodiment, the amounts per dose for the daily or weekly co-administration are from about 100 to about 400 mg of the HCV NS3/4a protease inhibitor and from about 40 to about 100 mg of the compound of formula (II).

Exemplary combinations of the HCV NS3/4a protease inhibitor (mg)/compound of formula (II) (mg) for twice daily dosage include 50/100, 100/100, 150/100, 200/100, 250/100, 300/100, 350/100, 400/100, 450/100, 50/133, 100/133, 150/133, 200/133, 250/133, 300/133, 50/150, 100/150, 150/150, 200/150, 250/150, 50/200, 100/200, 150/200, 200/200, 250/200, 300/200, 50/300, 80/300, 150/300, 200/300, 250/300, 300/300, 200/600, 400/600, 600/600, 800/600, 1000/600, 200/666, 400/666, 600/666, 800/666, 1000/666, 1200/666, 200/800, 400/800, 600/800, 800/800, 1000/800, 1200/800, 200/1200, 400/1200, 600/1200, 800/1200, 1000/1200, and 1200/1200. Other exemplary combinations of the HCV NS3/4a protease inhibitor (mg)/compound of formula (II) (mg) for twice daily dosage include 1200/400, 800/400, 600/400, 400/200, 600/200, 600/100, 500/100, 400/50, 300/50, and 200/50.

It will be understood, however, that specific dose level and frequency of dosage for any particular patient may be varied and will depend upon a variety of factors including the activity of the specific compound employed, the metabolic stability and length of action of that compound; the age, body weight, general health, sex and diet of the patient; mode and time of administration, rate of excretion, drug combination, the severity of the particular condition, and the type of patient undergoing therapy.

In one embodiment of the present invention there is provided an article of manufacture comprising a composition effective to treat an HCV infection or to inhibit the NS3 protease of HCV; and packaging material comprising a label which indicates that the composition can be used to treat infection by the hepatitis C virus; wherein the composition comprises the combination as described herein.

Another embodiment of the present invention concerns a kit or container comprising a combination according to the invention combining an HCV NS3/4a protease inhibitor or a pharmaceutically acceptable salt thereof, and the compound of formula (II) or a pharmaceutically acceptable salt thereof, in an amount effective for use as a standard or reagent in a test or assay for determining the ability of potential pharmaceuticals to inhibit HCV NS3/4a protease, HCV growth, or both. This aspect of the invention may find its use in pharmaceutical research programs.

The combinations of the present invention can be used in high-throughput target-analyte assays such as those for measuring the efficacy of said combination in HCV treatment.

### EXAMPLES

The following examples are meant to be illustrative of the present invention. These examples are presented to exemplify the invention and are not to be construed as limiting the scope of the invention.

### Example 1

### In Vitro Metabolic Blocking of HCV NS3/4a Protease Inhibitors by Compound of Formula (II)

Different HCV NS3/4a protease inhibitors were tested in a metabolic blocking experiment using 3 μM test compound together with 10 μM of compound of formula (II) acting as a cytochrome P450 inhibitor (or booster).

Test compounds and compound of formula (II) were added to human liver microsomes (protein concentration 1 mg/ml) suspended in potassium phosphate buffer (pH=7.4), to get final reaction mixture concentrations of 3 μM test compound and 10 μM of compound of formula (II). In the non-boosted parallel reactions, compound of formula (II) was not added. Boiled human liver microsomes were used for blank experiments. After addition (in a 1:3 ratio) of a cofactor mixture consisting of β-nicotinamide adenine dinucleotide phosphate (β-NADP, 0.5 mg/ml, 653.2 μM), D-Glucose-6-phosphate (2 mg/ml, 7.1 mM), Glucose-6-phosphate dehydrogenase (1.5 U/ml) in 2% NaHCO₃, the reaction mixture was incubated at 37° C. for 30 or 120 minutes after which the reaction was stopped by increasing the temperature to 95° C. Test compound concentrations were determined using HPLC-MS.

Results are summarized in the tables 3 and 4 below. Values are percentages of test compound detected after the indicated incubation times as compared to the initial test compound concentration. In Table 3, each value is the mean of the results of two independent experiments. In Table 4, each value is the result of an independent experiment.

TABLE 3

| | Incubation time: 30′ | | Incubation time: 120′ | |
|---|---|---|---|---|
| | % Detected Compound | | % Detected Compound | |
| | No | Compound of | | Compound of |
| Compound nr. | Booster | formula (II) | No Booster | formula (II) |
| 7 | 141 | 166 | 48 | 160 |
| 21 | 109 | 151 | 25 | 135 |
| 18 | 42 | 131 | 0 | 196 |
| BILN-2061 | 129 | 163 | 76 | 128 |
| 26 | 16 | 128 | 0 | 101 |

TABLE 4

| | Incubation time: 30′ | | Incubation time: 120′ | |
|---|---|---|---|---|
| | % Detected Compound | | % Detected Compound | |
| | No | Compound of | | Compound of |
| Compound nr. | Booster | formula (II) | No Booster | formula (II) |
| 13 | 90 | 43 | 89 | 51 |
| 13 | 87 | 83 | 82 | 95 |

The experiment shows an almost complete blocking of test compound (3 μM) metabolisation by addition of 10 μM of compound of formula (II).

1. A combination comprising
(a) an HCV NS3/4a protease inhibitor or a pharmaceutically acceptable salt thereof, wherein the HCV NS3/4a protease inhibitor is metabolized by cytochrome P450, and is selected from BILN-2061, VX-950, SCH 503034, ITMN-191, and the compound of formula (I) 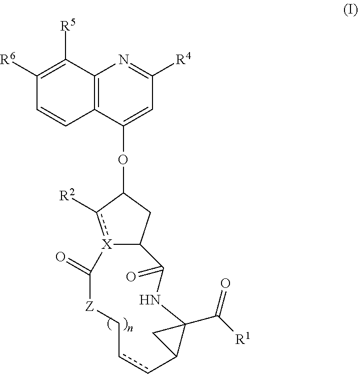
the salts and stereoisomeric forms thereof, wherein
each dashed line (represented by - - - - -) represents an optional double bond;
X is N, CH and where X bears a double bond it is C;
Z is —NR³—, —CR^{3a}R^{3b}—;
R¹ is —OR⁷, —NH—SO₂R⁸;
R² is hydrogen, and where X is C or CH, R² may also be C₁₋₆alkyl;
R³ is hydrogen, C₁₋₆alkyl, C₁₋₆alkoxyC₁₋₆alkyl, C₃₋₇cycloalkyl;
R^{3a} and R^{3b} are hydrogen or C₁₋₆alkyl; or R^{3a} and R^{3b} taken together may form a C₃₋₇cycloalkyl ring;
R⁴ is aryl or Het;
n is 3, 4, 5, or 6;
R⁵ represents hydrogen, halo, C₁₋₆alkyl, hydroxy, C₁₋₆alkoxy, polyhaloC₁₋₆alkyl, phenyl, or Het;
R⁶ represents C₁₋₆alkoxy, mono- or diC₁₋₆alkylamino;
R⁷ is hydrogen; aryl; Het; C₃₋₇cycloalkyl optionally substituted with C₁₋₆alkyl; or C₁₋₆alkyl optionally substituted with C₃₋₇cycloalkyl, aryl or with Het;
R⁸ is aryl; Het; C₃₋₇cycloalkyl optionally substituted with C₁₋₆alkyl; or C₁₋₆alkyl optionally substituted with C₃₋₇cycloalkyl, aryl or with Het;
aryl as a group or part of a group is phenyl optionally substituted with one, two or three substituents selected from halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkyl-amino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkyl-piperazinyl, 4-C₁₋₆alkylcarbonyl-piperazinyl, and morpholinyl; wherein the morpholinyl and piperidinyl groups may be optionally substituted with one or with two C₁₋₆alkyl radicals;
Het as a group or part of a group is a 5 or 6 membered saturated, partially unsaturated or completely unsaturated heterocyclic ring containing 1 to 4 heteroatoms each independently selected from nitrogen, oxygen and sulfur, said heterocyclic ring being optionally condended with a benzene ring; and Het as a whole being optionally substituted with one, two or three substituents each independently selected from the group consisting of halo, hydroxy, nitro, cyano, carboxyl, C₁₋₆alkyl, C₁₋₆alkoxy, C₁₋₆alkoxyC₁₋₆alkyl, C₁₋₆alkylcarbonyl, amino, mono- or di-C₁₋₆alkylamino, azido, mercapto, polyhaloC₁₋₆alkyl, polyhaloC₁₋₆alkoxy, C₃₋₇cycloalkyl, pyrrolidinyl, piperidinyl, piperazinyl, 4-C₁₋₆alkylpiperazinyl, 4-C₁₋₆alkylcarbonylpiperazinyl, and morpholinyl; wherein the morpholinyl and piperidinyl groups may be optionally substituted with one or with two C₁₋₆alkyl radicals; and
(b) a compound of the formula (II), 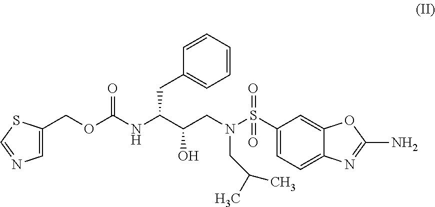
or a pharmaceutically acceptable salt thereof.

2. The combination according to claim 1 wherein the HCV NS3/4a protease inhibitor has the formula (III): 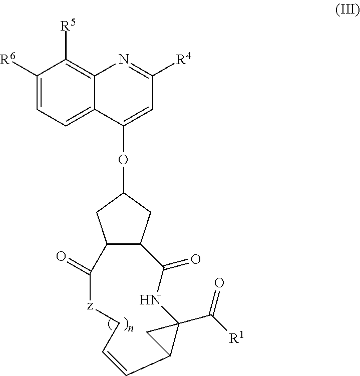
and wherein R¹; Z; R⁴, R⁵, R⁶, n are as recited in claim 1.

3. The combination according to claim 2 wherein the HCV NS3/4a protease inhibitor is selected from

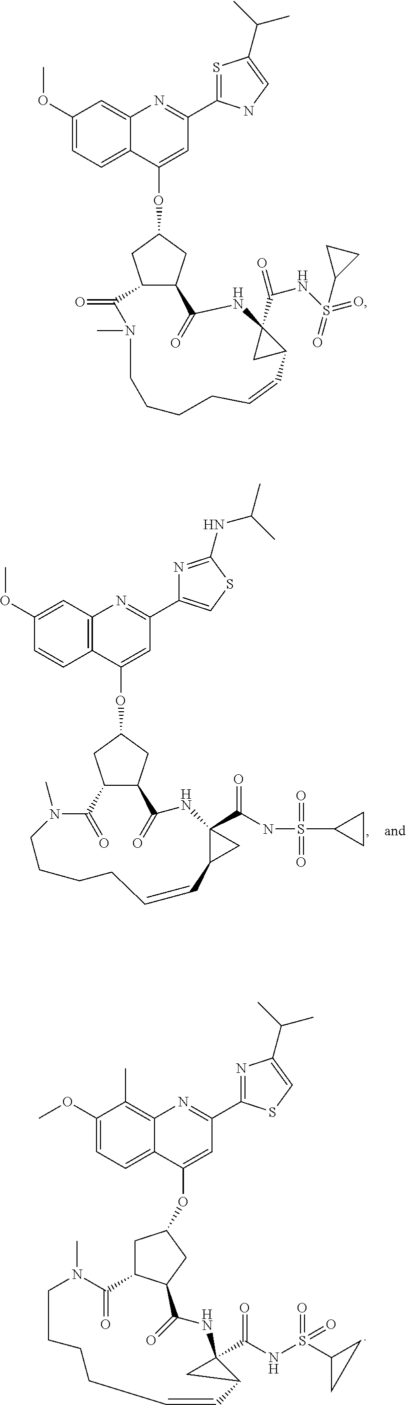

4. The combination according to claim 1 wherein the HCV NS3/4a protease inhibitor has the formula (IV): 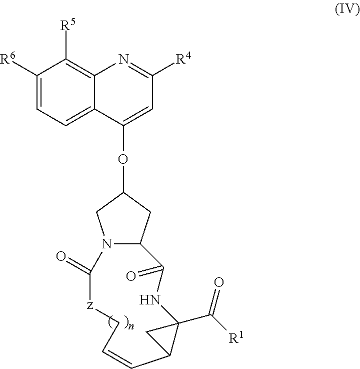
and wherein R¹; Z; R⁴, R⁵, R⁶, n are as recited in claim 1.

5. The combination according to claim 4 wherein the HCV NS3/4a protease inhibitor is selected from

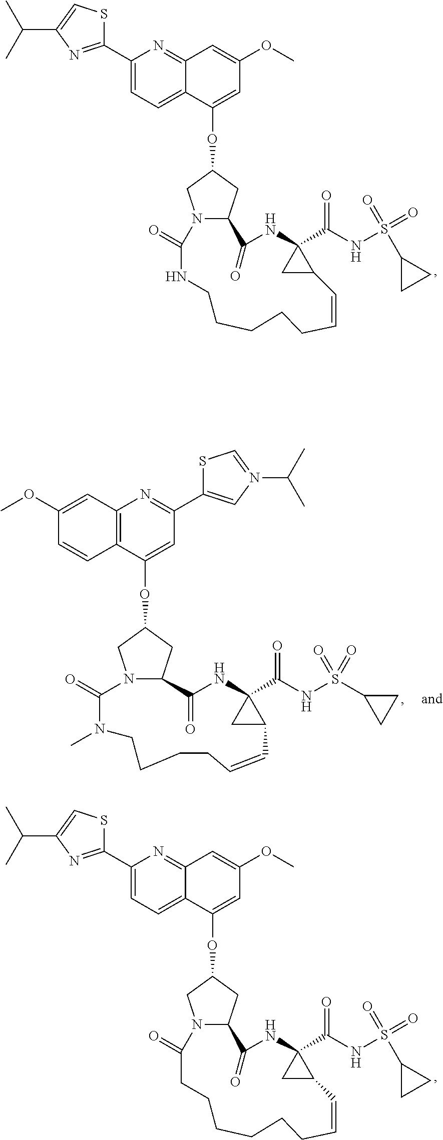

6. The combination according to any one of claims 1-5, wherein the amount of the compound of formula (II), or a pharmaceutically acceptable salt thereof, is sufficient to clinically improve the bioavailability of the HCV NS3/4a protease inhibitor relative to the bioavailability when said HCV NS3/4a protease inhibitor is administered alone.

7. The combination according to any one of claims 1-5, wherein the amount of the compound of formula (II), or a pharmaceutically acceptable salt thereof, is sufficient to increase at least one of the pharmacokinetic variables of the HCV NS3/4a protease inhibitor selected from t_{1/2}, Cₘᵢₙ, Cₘₐₓ, Cₛₛ, AUC at 12 hours, or AUC at 24 hours, relative to said at least one pharmacokinetic variable when the HCV NS3/4a protease inhibitor is administered alone.

8. The combination according to any one of claims 1-5, further comprising another HCV antiviral selected from an HCV polymerase inhibitor, an HCV protease inhibitor, an inhibitor of another target in the HCV life cycle, and immunomodulatory agent, an antiviral agent, and combinations thereof.

9. A pharmaceutical composition comprising a combination according to any one of claims 1-5 and a pharmaceutically acceptable excipient.

10. A product containing an HCV NS3/4a protease inhibitor or a pharmaceutically acceptable salt thereof, wherein the HCV NS3/4a protease inhibitor is metabolized by cytochrome P450, and is selected from BILN-2061, VX-950, SCH 503034, ITMN-191, and the compound of formula (I) as claimed in claim 1; and the compound of formula (II) or a pharmaceutically acceptable salt thereof; as a combined preparation for simultaneous, separate or sequential use in HCV therapy.

11. The combination according to any one of claims 1-5 for use as a medicament.

12. Use of the combination according to any one of claims 1-5 for the manufacture of a medicament for the treatment of HCV.

13. Use of the compound of formula (II) as claimed in claim 1, or a pharmaceutically acceptable salt thereof; as an improver of at least one of the pharmacokinetic variables of an HCV NS3/4a protease inhibitor selected from t_{1/2}, Cₘᵢₙ, Cₘₐₓ, Cₛₛ, AUC at 12 hours, or AUC at 24 hours; with the proviso that said use is not practised in the human or animal body.

14. An article of manufacture comprising a composition effective to treat an HCV infection or to inhibit the NS3 protease of HCV; and packaging material comprising a label which indicates that the composition can be used to treat infection by the hepatitis C virus; wherein the composition comprises the combination according to any one of claims 1-5.

15. A process for preparing the combination according to any one of claims 1-5, comprising the step of combining an HCV NS3/4a protease inhibitor or a pharmaceutically acceptable salt thereof; and the compound of formula (II) or a pharmaceutically acceptable salt thereof.

16. A method for treating HCV infection comprising administering to a patient in need of such treatment a combination according to any one of claims 1-5, comprising a therapeutically effective amount of each component of said combination.

17. A method for improving the bioavailability of a HCV NS3/4a protease inhibitor comprising administering to an individual in need of such improvement a combination according to any one of claims 1-5, comprising a therapeutically effective amount of each component of said combination.

18. The method according to any one of claims 16-17, wherein the HCV NS3/4a protease inhibitor or a pharmaceutically acceptable salt thereof; and the compound of formula (II) as claimed in claim 1 or a pharmaceutically acceptable salt thereof; are in separate dosage forms, or in a single dosage form.

19. The method according to claim 18 wherein the separate dosage forms are administered about simultaneously.
